(19) United States
(12) Patent Application Publication
(10) Pub. No.: US 2018/0055557 A1
(43) Pub. Date: Mar. 1, 2018

(54) ABLATION MANAGEMENT

(71) Applicant: RAINBOW MEDICAL LTD., Herzliya (IL)

(72) Inventors: Yossi GROSS, Moshav Mazor (IL); Yehuda ZADOK, Kiryat Ono (IL)

(73) Assignee: RAINBOW MEDICAL LTD., Herzliya (IL)

(21) Appl. No.: 15/254,494

(22) Filed: Sep. 1, 2016

(51) Int. Cl. A61B 18/12; A61B 18/14

(52) U.S. Cl. CPC A61B 18/1233; A61B 2018/00267; A61B 18/1492

(57) ABSTRACT

During a dual-signal period, a dual electric signal is applied at a site of a nerve of a subject, and during an ablation-signal period that is separate from the dual-signal period, an ablation electric signal is applied at the site. The dual electric signal has an excitation component and an ablation component, and the ablation electric signal has the ablation component but not the excitation component. Other embodiments are also described.

### FIELD OF THE INVENTION

Applications of the present invention relate generally to ablation of tissue. Some applications of the present invention relate more specifically to controlled ablation of nerve tissue.

### BACKGROUND

Hypertension is a prevalent condition in the general population, particularly in older individuals. Sympathetic nervous pathways, such as those involving the renal nerve, are known to play a role in regulating blood pressure. Ablation of renal nerve tissue from the renal artery is a known technique for treating hypertension. Renal denervation (RDN) is typically performed by advancing an electrode into the renal artery of a subject, and driving the electrode to apply ablation current, via the wall of the artery, to the renal nerve associated with the artery.

### SUMMARY OF THE INVENTION

The techniques described herein are hypothesized by the inventors to facilitate (i) identification of sites within the renal artery at which application of ablation current will effectively ablate the renal nerve, and/or (ii) monitoring of the progress of the ablation. Other techniques hypothesized to facilitate site-identification and/or progress-monitoring have been previously described by the inventors in PCT application publications WO 2014/068577 to Gross, and WO 2015/170281 to Gross et al., which may provide useful background to the techniques described herein, and which are incorporated herein by reference.

In the above-referenced publications, techniques are described in which excitatory current is applied to the renal nerve before and/or after ablative current is applied, in order to facilitate (i) identification of sites within the renal artery at which application of the ablation current will effectively ablate the renal nerve, and/or (ii) monitoring of the progress of the ablation.

Techniques described in the present application relate to exciting a site on the renal nerve generally at the same time as ablating that site (e.g., using the same electrode). For example, a dual electric signal is applied, that frequently alternates between an excitation component and an ablation component. Alternatively, the dual electric signal may be a complex or modulated signal that has both the excitation component and the ablation component. The effect of the excitation component on a parameter (e.g., blood pressure) of the subject is determined, and is used to facilitate (i) identification of sites within the renal artery at which application of the ablation current will effectively ablate the renal nerve, and/or (ii) monitoring of the progress of the ablation.

The techniques described herein are hypothesized by the inventors to have certain advantages over the techniques described in the above-referenced PCT publications. For example, because the excitation component is applied during ablation, it is not necessary to interrupt the ablation process, thereby reducing the time required. Because multiple excitations are typically required at each site, and because several sites per subject are typically screened and/or treated, the cumulative reduction in time for the overall procedure may be significant. For some applications, the techniques described herein facilitate performing a greater number and/or frequency of excitations, thus increasing the accuracy of ablation-progress monitoring.

There is therefore provided, in accordance with an application of the present invention, a method, including:

during a dual-signal period, applying, via an electrode disposed at a site of a nerve of a subject, a dual electric signal that has (i) an excitation component that has a frequency of 20-500 Hz, and (ii) an ablation component that has a frequency of 5 kHz-1 GHz; and

during an ablation-signal period that is separate from the dual-signal period, applying, via the electrode disposed at the site, an ablation electric signal that has the ablation component but not the excitation component.

In an application, the dual electric signal is a dual electric signal that alternates between the excitation component and the ablation component more frequently than once every two seconds, and applying the dual electric signal during the dual-signal period includes applying, during the dual-signal period, the dual electric signal that alternates between the excitation component and the ablation component more frequently than once every two seconds.

In an application, the dual electric signal is a complex signal that contains the excitation component and the ablation component, and applying the dual electric signal during the dual-signal period includes applying, during the dual-signal period, the complex signal that contains the excitation component and the ablation component.

In an application, the excitation component has a square waveform, and applying the dual electric signal that has the excitation component includes applying the dual electric signal that has the excitation component that has the square waveform.

In an application, the ablation component has a sinusoid waveform, and applying the dual electric signal that has the ablation component includes applying the dual electric signal that has the ablation component that has the sinusoid waveform.

In an application, applying the dual electric signal that has the excitation component includes applying the dual electric signal such that the excitation component has a power of less than 1 W.

In an application, applying the dual electric signal that has the ablation component includes applying the dual electric signal such that the ablation component has a power of 1-7 W.

In an application, the method further includes detecting a change in a value of a parameter of a subject during the first dual-signal period, and at least in part responsively to the detected change, configuring one or more characteristics selected from the group consisting of: a duration of the ablation-signal period, a frequency of the ablation electric signal, and an amplitude of the ablation electric signal.

In an application:

the dual-signal period is a first dual-signal period, and applying the dual electric signal includes applying a first application of the dual electric signal,

the ablation-signal period is subsequent to the first dual-signal period, and

the method further includes:
detecting a change in a value of a parameter of a subject during the first dual-signal period;
during a second dual-signal period that is subsequent to the ablation-signal period, applying, via the electrode disposed at the site, a second application of the dual electric signal; and
at least in part responsively to the detected change, configuring one or more characteristics selected from the group consisting of: a duration of the second dual-signal period, a frequency of the dual electric signal during the second application of the dual electric signal, and an amplitude of the dual electric signal during the second application of the dual electric signal.

In an application, the selected characteristic is the duration of the second dual-signal period, and configuring the one or more characteristics includes configuring the duration of the second dual-signal period.

In an application, detecting the change in the value of the parameter includes timing a duration of the first dual-signal period until the detected change reaches a pre-determined magnitude, and configuring the duration of second dual-signal period includes configuring the duration of the second dual-signal period according to the duration of the first dual-signal period.

In an application:

the ablation-signal period is subsequent to the dual-signal period,

the method further includes detecting a change in a value of a parameter of a subject during the dual-signal period, and

applying the ablation electric signal includes applying the ablation electric signal in response to the change in the value of the parameter during the dual-signal period.

In an application:

the dual-signal period is a first dual-signal period, and applying the dual electric signal includes applying a first application of the dual electric signal,

the ablation-signal period is a first ablation-signal period, and applying the ablation electric signal includes applying a first application of the ablation electric signal, and

the method further includes:
during a second dual-signal period that is subsequent to the first ablation-signal period, applying, via the electrode disposed at the site, a second application of the dual electric signal;
detecting a change in the value of the parameter during the second dual-signal period; and
in response to the change in the value of the parameter during the second dual-signal period, during a second ablation-signal period, applying, via the electrode disposed at the site, a second application of the ablation electric signal.

In an application, applying the second application of the ablation electric signal in response to the change in the value of the parameter during the second dual-signal period includes applying the second application of the ablation electric signal in response to a difference between (i) the change in the value of the parameter during the first dual-signal period, and (ii) the change in the value of the parameter during the second dual-signal period.

In an application:

the dual-signal period is a first dual-signal period, and applying the dual electric signal includes applying a first application of the dual electric signal,

the ablation-signal period is a first ablation-signal period, and applying the ablation electric signal includes applying a first application of the ablation electric signal, and

the method further includes:
during a second dual-signal period that is subsequent to the ablation-signal period, applying, via the electrode disposed at the site, a second application of the dual electric signal;
detecting a change in the value of the parameter during the second dual-signal period; and
in response to the change in the value of the parameter during the second dual-signal period, moving the electrode to another site of the nerve of the subject.

In an application, moving the electrode to the other site of the nerve of the subject in response to the change in the value of the parameter during the second dual-signal period includes moving the electrode to the other site of the nerve of the subject in response to a difference between (i) the change in the value of the parameter during the first dual-signal period, and (ii) the change in the value of the parameter during the second dual-signal period.

There is further provided, in accordance with an application of the present invention, apparatus for use with a blood vessel of a subject, the apparatus including:

a longitudinal member that has a proximal portion and a distal portion, including an electrode disposed at the distal portion, the distal portion being transluminally advanceable to the blood vessel; and

a control unit, in electrical communication with the electrode, and including circuitry, configured to:
during a dual-signal period, drive the electrode to apply a dual electric signal that has (i) an excitation component that has a frequency of 20-500 Hz, and (ii) an ablation component that has a frequency of 5 kHz-1 GHz; and
during an ablation-signal period that is separate from the dual-signal period, drive the electrode to apply an ablation electric signal that has the ablation component but not the excitation component.

In an application, the dual electric signal is a dual electric signal that alternates between the excitation component and the ablation component more frequently than once every two seconds, and the control unit is configured to, during the dual-signal period, drive the electrode to apply the dual electric signal that alternates between the excitation component and the ablation component more frequently than once every two seconds.

In an application, the dual electric signal is a complex signal that contains the excitation component and the ablation component, and the control unit is configured to, during the dual-signal period, drive the electrode to apply the complex signal that contains the excitation component and the ablation component.

In an application, the control unit is configured to configure the excitation component to have a square waveform.

In an application, the control unit is configured to configure the ablation component to have a sinusoid waveform.

In an application, the control unit is configured to apply the excitation component at a power of less than 1 W.

In an application, activating the control unit to apply the ablation component at a power of 1-7 W.

In an application, the apparatus is for use with a blood pressure sensor, and:

the control unit includes a blood-pressure-sensor interface that provides communication between the control unit and the blood pressure sensor; and

the circuitry is configured to receive blood-pressure information from the blood pressure sensor during the dual-signal period.

In an application, the apparatus further includes the blood pressure sensor, and the blood-pressure sensor is in electrical communication with the control unit via the blood-pressure-sensor interface.

In an application:

the dual-signal period is a first dual-signal period, and the dual electric signal applied during the first dual-signal period is a first application of the dual electric signal, and

the circuitry is configured:
such that the ablation-signal period is subsequent to the first dual-signal period,
to apply, during a second dual-signal period that is subsequent to the ablation-signal period, a second application of the dual electric signal,
to identify a change in a blood-pressure value of the subject during the first dual-signal period,
at least in part responsively to the change, to configure one or more characteristics selected from the group consisting of: a duration of the second dual-signal period, a frequency of the dual electric signal during the second application of the dual electric signal, and an amplitude of the dual electric signal during the second application of the dual electric signal.

In an application, the circuitry is configured to identify a change in a blood-pressure value of the subject during the first dual-signal period, and at least in part responsively to the change, to configure one or more characteristics selected from the group consisting of: a duration of the ablation-signal period, a frequency of the ablation electric signal, and an amplitude of the ablation electric signal.

There is further provided, in accordance with an application of the present invention, a method, including:

advancing an electrode into a blood vessel of a subject; and

while the electrode is disposed at a site within the blood vessel, activating a control unit to drive the electrode to apply a dual electric signal that has (i) an excitation component that has a frequency of 20-500 Hz, and (ii) an ablation component that has a frequency of 5 kHz-1 GHz, and that alternates between the excitation component and the ablation component more frequently than once every two seconds.

In an application, the excitation component has a square waveform, and activating the control unit to drive the electrode to apply the dual electric signal that has the excitation component includes activating the control unit to drive the electrode to apply the dual electric signal that has the excitation component that has the square waveform.

In an application, the ablation component has a sinusoid waveform, and activating the control unit to drive the electrode to apply the dual electric signal that has the ablation component includes activating the control unit to drive the electrode to apply the dual electric signal that has the ablation component that has the sinusoid waveform.

In an application, activating the control unit to drive the electrode to apply the dual electric signal that has the excitation component includes activating the control unit to drive the electrode to apply the dual electric signal such that the excitation component has a power of less than 1 W.

In an application, activating the control unit to drive the electrode to apply the dual electric signal that has the ablation component includes activating the control unit to drive the electrode to apply the dual electric signal such that the ablation component has a power of 1-7 W.

In an application, the method further includes, while the electrode remains disposed at the site, and subsequently to the application of the dual electric signal, activating the control unit to drive the electrode to apply an ablation electric signal that has the ablation component but not the excitation component.

In an application, activating the control unit to drive the electrode to apply the dual electric signal includes activating the control unit to drive the electrode to apply a first application of the dual electric signal, and the method further includes, subsequently to the application of the ablation electric signal, and while the electrode remains disposed at the site, activating the control unit to drive the electrode to apply a second application of the dual electric signal.

In an application:

the dual electric signal alternates between bursts of the excitation component and bursts of the ablation component such that, during a dual-signal period (i) in which the dual signal is applied, (ii) that has a duration and, (iii) that contains an equal number of excitation-component bursts and ablation-component bursts, the ablation-component bursts cumulatively have an ablation-component duration of 70-95 percent of a duration of the dual-signal period, and

activating the control unit to apply the dual electric signal includes activating the control unit to apply the dual electric signal that alternates between bursts of the excitation component and bursts the ablation component such that, during the dual-signal period, the ablation-component bursts cumulatively have the ablation-component duration of 70-95 percent of the duration of the dual signal period.

In an application:

the dual electric signal alternates between bursts of the excitation component and bursts of the ablation component such that, during a dual-signal period that has a duration and contains an equal number of excitation-component bursts and ablation-component bursts, the excitation-component bursts cumulatively have an excitation-component duration of 5-30 percent of a duration of the dual-signal period, and

activating the control unit to apply the dual electric signal includes activating the control unit to apply the dual electric signal that alternates between bursts of the excitation component and bursts of the ablation component such that, during the dual-signal period, the excitation-component bursts cumulatively have an excitation-component duration of 5-30 percent of the duration of the dual signal period.

There is further provided, in accordance with an application of the present invention, a method, including:

advancing an electrode into a blood vessel of a subject; and

while the electrode is disposed at a site within the blood vessel, activating a control unit to:
during a dual-signal period, drive the electrode to apply a dual electric signal that has (i) an excitation component that has a frequency of 20-500 Hz, and (ii) an ablation component that has a frequency of 5 kHz-1 GHz, and
during an ablation-signal period that is separate from the dual-signal period, drive the electrode to apply an ablation electric signal that has the ablation component but not the excitation component.

In an application, the dual electric signal is a dual electric signal that alternates between the excitation component and the ablation component more frequently than once every two seconds, and activating the control unit to apply the dual electric signal during the dual-signal period includes activating the control unit to, during the dual-signal period, apply the dual electric signal that alternates between the excitation component and the ablation component more frequently than once every two seconds.

In an application, the dual electric signal is a complex signal that contains the excitation component and the ablation component, and activating the control unit to apply the dual electric signal during the dual-signal period includes activating the control unit to, during the dual-signal period, apply the complex signal that contains the excitation component and the ablation component.

In an application, the excitation component has a square waveform, and activating the control unit to drive the electrode to apply the dual electric signal that has the excitation component includes activating the control unit to drive the electrode to apply the dual electric signal that has the excitation component that has the square waveform.

In an application, the ablation component has a sinusoid waveform, and activating the control unit to drive the electrode to apply the dual electric signal that has the ablation component includes activating the control unit to drive the electrode to apply the dual electric signal that has the ablation component that has the sinusoid waveform.

In an application, activating the control unit to drive the electrode to apply the dual electric signal that has the excitation component includes activating the control unit to drive the electrode to apply the dual electric signal such that the excitation component has a power of less than 1 W.

In an application, activating the control unit to drive the electrode to apply the dual electric signal that has the ablation component includes activating the control unit to drive the electrode to apply the dual electric signal such that the ablation component has a power of 1-7 W.

There is further provided, in accordance with an application of the present invention, a method, including:

during a first period, applying at a site in a blood vessel of a subject, (i) an excitation electric signal that has a frequency of 20-500 Hz, and (ii) ultrasound energy; and

during a second period that is separate from the first period, applying at the site, ultrasound but not the excitation electric signal.

There is further provided, in accordance with an application of the present invention, apparatus for use with a blood vessel of a subject, the apparatus including:

a longitudinal member that has a proximal portion and a distal portion, including, at the distal portion, an electrode and an ultrasound transducer, the distal portion being transluminally advanceable to the blood vessel; and

a control unit, in electrical communication with the electrode and the transducer, and including circuitry, configured to:
during a first period, (i) drive the electrode to apply an excitation electric signal that has a frequency of 20-500 Hz to tissue at a site in the blood vessel, and (ii) drive the transducer to apply ultrasound to the tissue at the site; and
during a second period that is separate from the first period, drive the transducer to apply ultrasound to the tissue at the site, but not drive the electrode to apply the excitation electric signal to the tissue at the site.

In an application, the ultrasound transducer is a high intensity focused ultrasound (HIFU) transducer.

The present invention will be more fully understood from the following detailed description of applications thereof, taken together with the drawings, in which:

### BRIEF DESCRIPTION OF THE DRAWINGS

FIG. 1 is a flow chart showing at least some steps in a technique for facilitating renal denervation (RDN) procedures, in accordance with some applications of the invention;

FIG. 2 is an annotated graph showing blood pressure changes during different periods of an RDN procedure, in accordance with some applications of the invention;

FIG. 3 is a schematic illustration of a system being used to facilitate an RDN procedure, in accordance with some applications of the invention; and

FIG. 4 is a schematic illustration of an intravascular tool, in accordance with some applications of the invention.

### DETAILED DESCRIPTION OF EMBODIMENTS

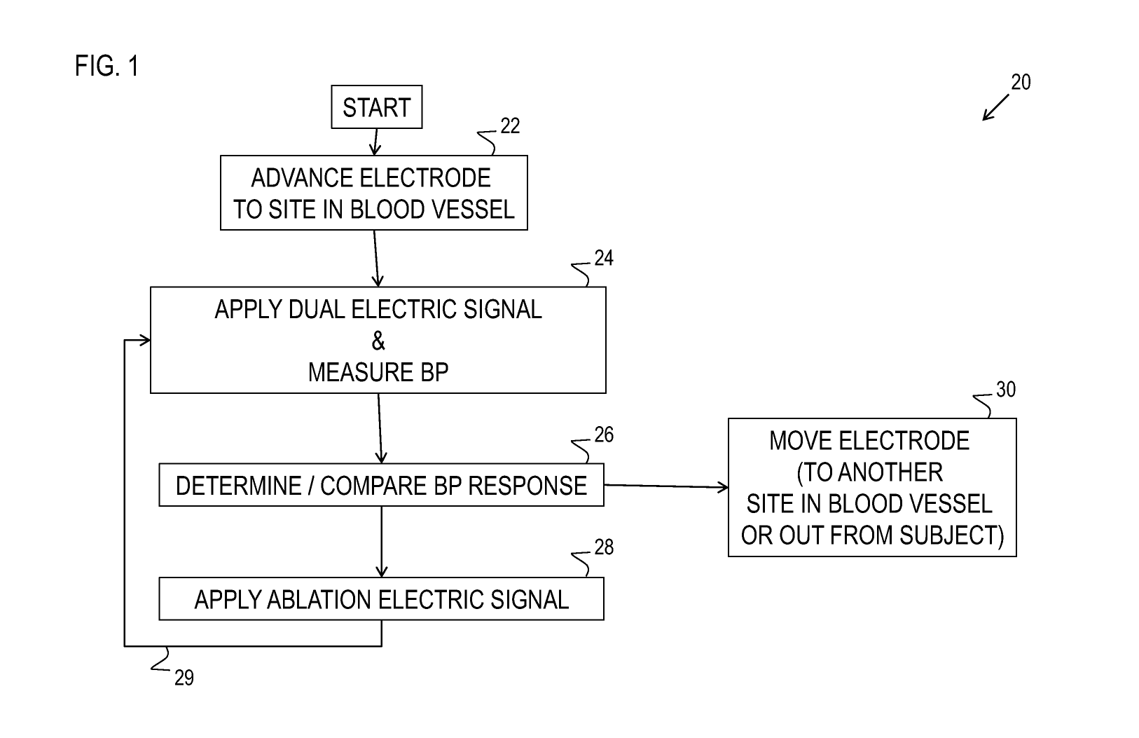

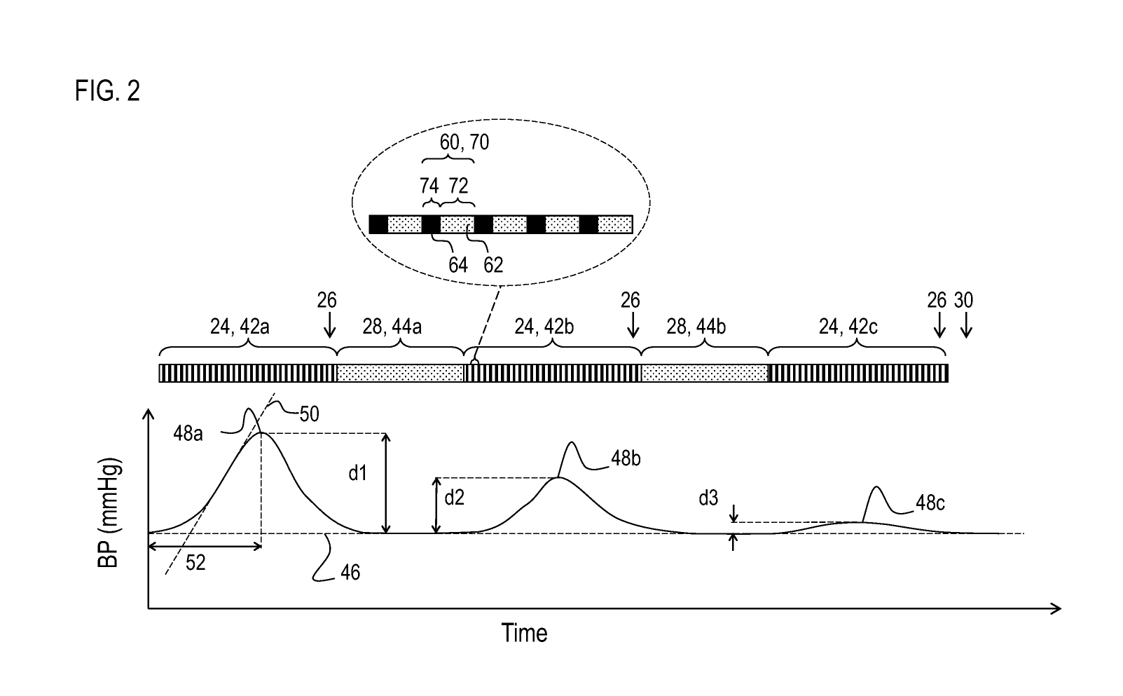

Reference is made to FIG. 1, which is a flow chart showing at least some steps in a technique 20 for facilitating renal denervation (RDN) procedures, in accordance with some applications of the invention. Reference is also made to FIG. 2, which is an annotated graph showing blood pressure changes during different periods of an RDN procedure, in accordance with some applications of the invention.

An electrode (typically disposed at a distal portion of a transluminal catheter) is advanced into the renal artery (step 22). The electrode is driven (e.g., by a control unit activated by the operator) to apply a dual electric signal to a site of the renal nerve (e.g., the site of the renal nerve closest to the location of the electrode within the renal artery) (step 24). The term “dual-signal period” is used herein to relate to the period between the start and the end of the application of the dual electric signal. The dual electric signal has an excitation component and an ablation component, e.g., described hereinbelow.

Blood pressure (BP; e.g., mean arterial pressure) of the subject is measured (shown as part of step 24), typically during the dual-signal period. The BP response to the excitation component (e.g., a BP change during the dual-signal period) is determined, such as by comparing a peak BP during the dual-signal period, to a rest BP (e.g., BP detected prior to step 24) (step 26). In response to the determined BP response, either (i) the electrode is driven to apply an ablation electric signal to the same site of the nerve (e.g., without moving the electrode) (step 28), or (ii) the electrode is moved-either to another site within the artery, or out of the subject (i.e., is removed from the subject) (step 30). The term “ablation-signal period” is used herein to relate to the period between the start and the end of the application of the ablation electric signal. The ablation electric signal has the ablation component but not the excitation component, e.g., as described hereinbelow.

If the ablation electric signal is applied, then following the application of the ablation electric signal, steps 24 and 26 are repeated (represented by arrow 29). This iterative process continues until the determined BP response to the preceding application of the dual electric signal is such that step 30 is performed.

The decision between proceeding to step 28 or to step 30 is made in response to the determined BP response to the preceding application of the dual electric signal (e.g., the change in BP during the preceding dual-signal period). This is now described in more detail:
The BP response to the first application of the dual electric signal (and thereby the first application of the excitation component) at a particular site is indicative of whether that site is appropriate for ablation of the renal nerve (e.g., whether the electrode is sufficiently close to the renal nerve). If the BP response to the first application of the dual electric signal at the site is low (e.g., below a pre-determined threshold value), then step 30 is performed. For example, if the site is the first site at which the dual electric signal is performed, then the electrode may be moved to another site within the renal artery, and the process is repeated at the other site. If one or more other sites have previously given low BP responses to application of the dual electrical signal, then the electrode may be withdrawn from the subject (e.g., it may be determined that the subject is not a suitable candidate for RDN).
The BP responses to subsequent applications of the dual electric signal at a particular site are indicative of the progress of the ablation process. If a BP response to an application of the dual electric signal at the site is smaller than the BP response to a preceding application of the dual electric signal at the same site, this typically indicates that nerve ablation has occurred since the preceding application of the dual electric signal. Typically, steps 24, 26 and 28 are repeated until a sufficiently small BP response to the dual electric signal is observed (e.g., indicating sufficient nerve ablation at that site), at which point the procedure moves to step 30. For some applications, the procedure moves to step 30 if the reduction in BP response since the preceding application of the dual electric signal is below a threshold reduction, e.g., irrespective of whether sufficient nerve ablation has occurred at that site (e.g., indicating that further applications of the dual electric signal are likely to be ineffective).

FIG. 2 shows a schematic line graph representing BP over time, during the performance of steps of technique 20 at a particular site of the renal nerve (e.g., a particular site within the renal artery). Juxtaposed with the line graph is a representation of the electric signal being applied to the site on the nerve.

The dual electric signal is applied, and BP is measured (step 24), defining a dual-signal period 42 (e.g., a first dual-signal period 42a). During period 42a, BP increases (e.g., from a rest BP value 46 to a peak BP value 48a). In step 26, this BP response d1 is determined, and the decision is made to move on to step 28, in which the ablation electric signal is applied, defining an ablation-signal period 44 (e.g., a first ablation-signal period 44a).

As described hereinabove, during ablation-signal period 44, the excitation component is not applied, and BP typically returns to rest value 46 during the ablation-signal period. Nonetheless, it is common for BP to begin to decrease from peak 48 even during dual-signal period 42. It is hypothesized by the inventors that this may be due to desensitization of the renal nerve to the excitation component of the dual electric signal, and/or compensation by the parasympathetic nervous system. It is further hypothesized by the inventors that this “rest” period provides an advantage to the techniques described herein (e.g., technique 20), compared to simply continuously applying the dual electric signal for the duration of the procedure: Providing a period without the excitation component (i.e., period 44) increases the accuracy of comparisons of BP response to the excitation component at different stages of the progressing ablation procedure, because during each dual-signal period 42 the BP response begins anew from baseline 46 (or close thereto). An alternative way of providing a rest period, that is within the scope of some applications of the invention, is to omit period 44 (i.e., step 28), e.g., applying no electric signal during rest periods between dual-signal periods 42. However, it is hypothesized that the inclusion of the ablation component (i.e., the ablation electric signal) during this rest period (as shown for technique 20) allows ablation to continue throughout the procedure, and thereby reduces the overall duration of the procedure.

Following period 44a, the dual electric signal is applied again, defining a second dual-signal period 42b, during which BP is measured (step 24). In the example shown in FIG. 2, the BP response d2 that is determined for period 42b is smaller than that determined for period 42a (e.g., peak 48b is smaller than peak 48a). As described hereinabove, this typically indicates that nerve ablation has occurred since the previous determination (e.g., during the latter part of period 42a and/or during period 44a). In the example shown in FIG. 2, in response to response d2, the procedure continues with a second ablation-signal period 44b, followed by a third dual-signal period 42c (i.e., another iteration of steps 28 and 24). For some applications, this continuing of the procedure is at least in part responsively to response d2 alone. (For example, if d2 had been sufficiently small, then the decision at step 26 may have been to move directly to step 30.) For some applications, this continuing of the procedure is at least in part responsively to responses d2 and d1, e.g., a comparison therebetween. (For example, if d2 had been sufficiently smaller than d1, then the decision at step 26 may have been to move directly to step 30.) In the example shown in FIG. 2, the response d3 in third dual-signal period 42c is even smaller than response d2, and at the subsequent step 26, the procedure moves to step 30 (e.g., at least in part responsively to response d3 alone, and/or at least in part responsively to response d2 and/or response d1).

For some applications, the first application of the excitation component at one or each site is performed by applying an application of an excitatory electric signal that has the excitation component but not the ablation component. That is, for some applications, the first iteration of step 24 comprises applying the excitatory electric signal instead of the dual electric signal. Alternatively, another step (not shown), in which the excitatory electric signal is applied and the BP response is detected, may be added between steps 22 and 24. By providing the first application of the excitation component without the ablation component, each site may be tested for its suitability for application of the ablative component prior to any application of the ablative component.

Reference is again made to FIG. 2. Step 26 is shown positioned generally toward the end of each dual-signal period 42 for the sake of simplicity. However, for some applications, the determination and/or comparison of the BP response may be performed earlier during period 42. For example, the determination and/or comparison may be based on a maximum BP increase-rate 50 for that period 42, or the rest-to-peak time 52 for that period 42. Alternatively, the determination and/or comparison of the BP response may be performed after the end of period 42 (e.g., during a gap between periods 42 and 44, or shortly after the start of period 44).

The ablative component typically (i) has a frequency of greater than 5 kHz and/or less than 1 GHz (e.g., 100-600 kHz, e.g., 200-600 kHz, e.g., 400-500 kHz, such as about 480 kHz), and/or (ii) is applied at a power of at least 1 W and/or less than 7 W (e.g., 1-7 W). Typically, the power at which the ablative component is applied is adjusted according to the temperature of the electrode and/or the surrounding tissue. Typically, the ablative component is controlled to maintain the electrode and/or the tissue at a target temperature (e.g., 60-72 degrees C., e.g., 68-71 degrees C., such as 70 degrees C.). For some applications, the ablative component has a sinusoid or mostly sinusoid waveform.

The excitation component typically (i) has a frequency of greater than 20 and/or less than 500 Hz (e.g., 20-500 Hz), and/or (ii) is applied at a power of less than 1 W (e.g., 0.05-1 W). In contrast to the ablative component, the power at which the excitation component is applied is typically independent of the temperature of the electrode and/or the surrounding tissue. Typically, the excitation component is controlled to maintain it at a constant current. For some applications, the excitation component has a rectangular waveform.

The dual electric signal may be achieved in several ways. For some applications, the dual electric signal frequently (e.g., (more than once every two seconds, such as more than once every second) alternates between the excitation component and the ablation component. That is, during the dual-signal period, ablation-component bursts 62 of the ablation component are intercalated with excitation-component bursts 64 of the excitation component, such that the effect of each component (e.g., with respect to the effect on the subject) is similar to as if it had been applied alone. For example, gaps between bursts 64 of the excitation component (i.e., during applications of the ablation component) may be sufficiently short that excitation of the nerve is still achieved, and gaps between bursts 62 of the ablation component (i.e., during applications of the excitation component) may be sufficiently short that ablation of the nerve is still achieved (e.g., such that the elevated temperature of the nerve tissue is maintained).

For applications in which the dual electric signal frequently alternates between bursts of the ablation and excitation components, the burst duration 74 of the excitation-component bursts 64 are typically shorter than the burst duration 72 of the ablation-component bursts. For example, burst duration 74 may be less than 60% (e.g., less than 50% less than 25%, such as less than 10%) and/or at least 1% as long as burst duration 72.

For applications in which the dual electric signal frequently alternates between bursts of the ablation and excitation components, the dual electric signal has a dual-burst period 60, which contains a complete excitation-component burst 64 and a temporally-adjacent complete ablation-component burst 62 (e.g., the dual-burst period (i) starts at the beginning of an excitation-component burst 64, and finishes at the end of the immediately-subsequent ablation-component burst 62, or (ii) starts at the beginning of an ablation-component burst 62, and finishes at the end of the immediately-subsequent excitation-component burst 64.) The dual-burst period has a dual-burst period duration 70. Burst duration 74 of excitation-component bursts 64 are typically less than 50% (e.g., less than 40%, e.g., less than 30% (such as 5-30%), e.g., less than 20%, e.g., less than 10%) and/or at least 1% as long as dual-burst period duration 70.

Typically, dual-burst period duration 70 is 500-2000 (e.g., 800-1200, such as 1000) ms. For some applications, burst duration 74 of bursts 64 of the excitation component is 50-500 (e.g., 50-200, such as 100) ms. For some applications, burst duration 72 of bursts 62 of the ablation component is 450-1950 (e.g., 700-1200, e.g., 800-1000, such as 900) ms.

For some applications, within excitation-component bursts 64, the excitation component is applied in a duty cycle of 1-40% (e.g., 5-20%, such as about 10%). That is, for some applications, the excitation component is “on” for 1-40% (e.g., 5-20%, such as about 10%) of burst duration 74 of each excitatory-component burst 64.

For some applications, rather than the dual electric signal being a signal that frequently alternates between bursts of the ablation and excitation components, the dual electric signal is a complex signal that contains both the excitation component and the ablation component.

For some applications (e.g., for those in which the ablation component is identical in both the dual electric signal and the ablation electric signal) switching between the ablation electric signal and the dual electric signal may be simply described as switching on and off of the excitation component. That is, for some applications, the ablation component is constant throughout steps 24, 26, and 28, and step 24 comprises adding the excitation component to the electric signal during the dual-signal period. For example:
For applications in which the dual electric signal is a complex or modulated signal that has both the excitation component and the ablation component, switching from the ablation electric signal to the dual electric signal may comprise creating the complex or modulated signal by adding the excitation component to the ablation electric signal during dual-signal period 42 (e.g., while keeping the ablation component unchanged).
For applications in which the dual electric signal comprises frequent alternating between the excitation and ablation component, switching from the ablation electric signal to the dual electric signal may comprise, during the dual-signal period, applying the excitation component during gaps in the application of the ablation component that were also present during the ablation-signal period (e.g., while keeping the ablation component unchanged).

For some applications, the ablation component is different during the dual-signal period than it is during the ablation-signal period. For example, gaps may be introduced into the ablation component during the dual-signal period to allow for application of the excitation component.

Each dual-signal period 42 typically has a duration of 10-300 s, such as 20-180 s, e.g., 30-120 s. Each ablation-signal period 44 typically has a duration of 10-300 s, such as 20-180 s, e.g., 20-60 s. For some applications, ablation-signal period 44 is shorter than dual-signal period.

For some applications, at least in part responsively to the detected BP response during a previous dual-signal period 42, the duration of a subsequent dual-signal period is changed. For example, if a first dual-signal period has a duration of 60 s but the peak BP during the response is reached after only 20 s, a subsequent dual-signal period may be shortened, e.g., so as to reduce the amount of unnecessary application of the excitation component, and/or to increase the duration of “rest” during the subsequent ablation-signal period. Conversely, if the BP were still increasing at the end of the first dual-signal period, the next dual-signal period may be lengthened, e.g., so as to identify the maximum BP achievable by stimulating the nerve site in its current state of ablation.

For some applications, at least in part responsively to the response during a previous dual-signal period 42, a characteristic (e.g., frequency, amplitude, duty cycle, waveform) of the dual electric signal (or a component thereof) is changed for a subsequent dual-signal period.

There is therefore provided, in accordance with some applications of the invention, a method, comprising: (a) advancing an electrode into a blood vessel of a subject; and (b) while the electrode is disposed at a site within the blood vessel, activating a control unit to drive the electrode to apply a dual electric signal that has (i) an excitation component that has a frequency of 20-500 Hz, and (ii) an ablation component that has a frequency of 5 kHz-1 GHz, and that alternates between the excitation component and the ablation component more frequently than once every two seconds.

There is further provided, in accordance with some applications of the invention: a method, comprising: (a) during a dual-signal period, applying, via an electrode disposed at a site of a nerve of a subject, a dual electric signal that has (i) an excitation component that has a frequency of 20-500 Hz, and (ii) an ablation component that has a frequency of 5 kHz-1 GHz; and (b) during an ablation-signal period that is separate from the dual-signal period, applying, via the electrode disposed at the site, an ablation electric signal that has the ablation component but not the excitation component. The dual electric signal may be (i) a dual electric signal that alternates between the excitation component and the ablation component more frequently than once every two seconds, or (ii) a complex signal that contains the excitation component and the ablation component.

For some applications, (i) the dual-signal period is a first dual-signal period, (ii) the ablation-signal period is subsequent to the first dual-signal period described above, and (iii) the method further comprises: (1) detecting a change in a value of a parameter (e.g., BP) of the subject during the first dual-signal period; (2) during a second dual-signal period that is subsequent to the ablation-signal period, applying, via the electrode disposed at the site, a second application of the dual electric signal; and (3) at least in part responsively to the detected change, configuring the duration of the second dual-signal period, the frequency of the dual electric signal applied during the second application of the dual electric signal, and/or the amplitude of the dual electric signal during the second application of the dual electric signal.

For some applications, (i) the dual-signal period is a first dual-signal period, (ii) the ablation-signal period is a first ablation-signal period, and (iii) the method further comprises: (1) during a second dual-signal period that is subsequent to the first ablation-signal period, applying, via the electrode disposed at the site, a second application of the dual electric signal; (2) detecting a change in the value of the parameter (e.g., BP) during the second dual-signal period; and (3) in response to the change in the value of the parameter during the second dual-signal period, during a second ablation-signal period, applying, via the electrode disposed at the site, a second application of the ablation electric signal.

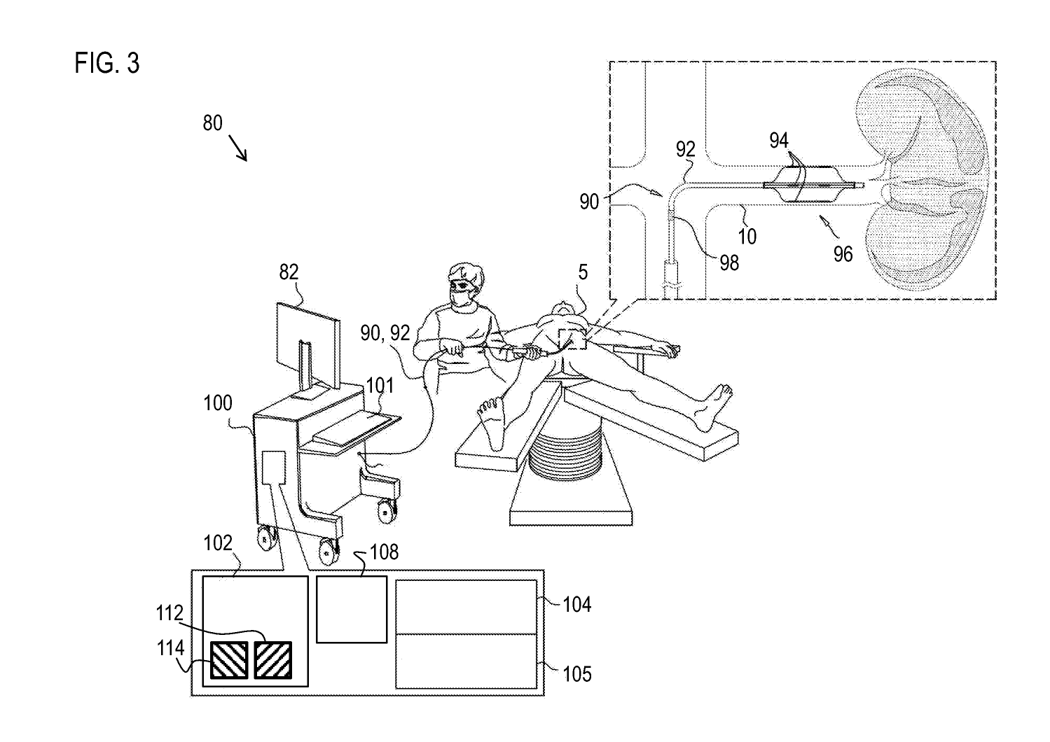

Reference is made to FIG. 3, which is a schematic illustration of a system 80 being used with a subject 5, in accordance with some applications of the invention. System 80 comprises an intravascular tool 90, a display 82, and a control unit 100. Tool 90 comprises a longitudinal member (e.g., a catheter) 92 that has a distal portion that comprises at least one electrode 94, and is transluminally advanceable into a blood vessel of subject 5, such as the renal artery 10 of the subject. Typically, tool 90 comprises a reversibly-expandable electrode device 96 on which the at least one electrode 94 is mounted. System 80 also comprises a blood pressure sensor 98, such as an intravascular pressure sensor (as shown), e.g., disposed on catheter 92. Alternatively, pressure sensor 98 may be an extracorporeal blood pressure sensor, such as a blood pressure cuff.

Control unit 100 comprises circuitry 102 (typically comprising at least one computer processor), a catheter interface (e.g., a port, such as a socket) 104 via which the control unit interfaces with tool 90, and a display interface (e.g., a port, such as a socket) 56 via which the control unit interfaces with display 82. Control unit 100 comprises a pressure-sensor interface (e.g., a port, such as a socket) 105 via which the control unit interfaces with pressure sensor 98. For some applications (e.g., for applications in which pressure sensor 98 is disposed on catheter 92), interface 105 and interface 104 may be integrated with each other, or may be subcomponents of a common interface. Alternatively (e.g., for applications in which pressure sensor 98 is a blood pressure cuff), interfaces 104 and 105 may be distinct from each other (e.g., may comprise separate connections for catheter 92 and sensor 98).

For some applications, control unit 100 also comprises at least one memory 108, which may be physically located within a common housing of the control unit, or may be located elsewhere and connected to circuitry 102 e.g., via a network. For some applications, memory 108 comprises one or more of the following: a semiconductor or solid state memory, magnetic tape, a removable computer diskette, a random access memory (RAM), a read-only memory (ROM), a rigid magnetic disk, and an optical disk.

Control unit 100 comprises at least one user input device 101, such as a mouse, keyboard, or trackball. For some applications, user input device 101 is integrated into display 82 as a touchscreen. It is to be understood that the scope of the invention includes the use of any appropriate input device known in the art. The operation of control unit 100 by the operator, described hereinbelow, are implemented via user input device 101.

For some applications, control unit 100 (e.g., circuitry 102 thereof) comprises an ablation-component generator 112 and an excitatory-component generator 114, which generate the ablation component and the excitation component, respectively. For some applications in which the dual electric signal is provided by frequently alternating between the ablation component and the excitation component, control unit 100 generates the dual electric signal by frequently alternating between activating generators 112 and 114. For some applications in which the dual electric signal is a complex signal, control unit 100 generates the dual electric signal by activating generators 112 and 114 generally at the same time.

For some applications, system 80 facilitates the performance of the techniques described herein. For some applications, one or more steps of the techniques described herein are at least in part automated by system 80. For example, as described hereinabove, frequently alternating between the ablation component and the excitation component (in order to provide the dual electric signal) may be performed automatically by control unit 100. Similarly, control unit 100 may, upon receiving an input via interface 104, automatically perform steps 28 and 24 in succession. For some applications, step 26 is also performed by control unit 100, such that the control unit performs iterations of steps 24, 26 and 28 until it determines that step 30 should be performed.

There is therefore provided, in accordance with some applications of the invention, apparatus for use with a blood vessel of a subject, the apparatus comprising:
(i) a longitudinal member that has a proximal portion and a distal portion, comprising an electrode disposed at the distal portion, the distal portion being transluminally advanceable to the blood vessel; and
(ii) a control unit, in electrical communication with the electrode, and comprising circuitry, configured to: (a) during a dual-signal period, drive the electrode to apply a dual electric signal that has (i) an excitation component that has a frequency of 20-500 Hz, and (ii) an ablation component that has a frequency of 5 kHz-1 GHz; and (b) during an ablation-signal period that is separate from the dual-signal period, drive the electrode to apply an ablation electric signal that has the ablation component but not the excitation component.

Reference is again made to FIGS. 1-3. It is to be noted that although blood pressure (e.g., mean arterial pressure, or systolic blood pressure) is used throughout this application as an example of a parameter of the subject that may be measured to facilitate the techniques described herein, other parameters (e.g., other blood pressure parameters, and/or non-blood-pressure parameters). In particular, another parameter may be used when the target nerve is a nerve other than the renal nerve. In general, the parameter that is used is a parameter affected by action potentials in the target nerve.

Reference is again made to FIGS. 1-3. It is to be noted that the techniques described herein may be applied to other nerves, such as nerves associated with blood vessels other than the renal artery (e.g., transvascularly), or nerves not associated with a particular blood vessel (e.g., by applying current from an extravascular electrode).

Reference is again made to FIGS. 1-3. In accordance with some applications of the invention, the techniques described hereinabove may be used in combination with, or to facilitate, techniques described by the inventors in PCT application publications WO 2014/068577 to Gross, and WO 2015/170281 to Gross et al., which are incorporated herein by reference.

Reference is again made to FIGS. 1-3. For some applications of the invention, techniques described hereinabove are modified to utilize ultrasound-energy based denervation (such as, but not limited to, high intensity focused ultrasound (HIFU)-based denervation). For example, for some applications of the invention, (1) during the ablation-signal period, ultrasound is applied instead of the ablation electric signal, and (2) during the dual-signal period, instead of applying a dual electric signal, simultaneous application of ultrasound and the excitation component is used, mutatis mutandis. That is, for some applications of the invention, ultrasound is applied throughout steps 24 and 28, while the excitation component (e.g., as an excitation electric signal) is applied only during step 24.

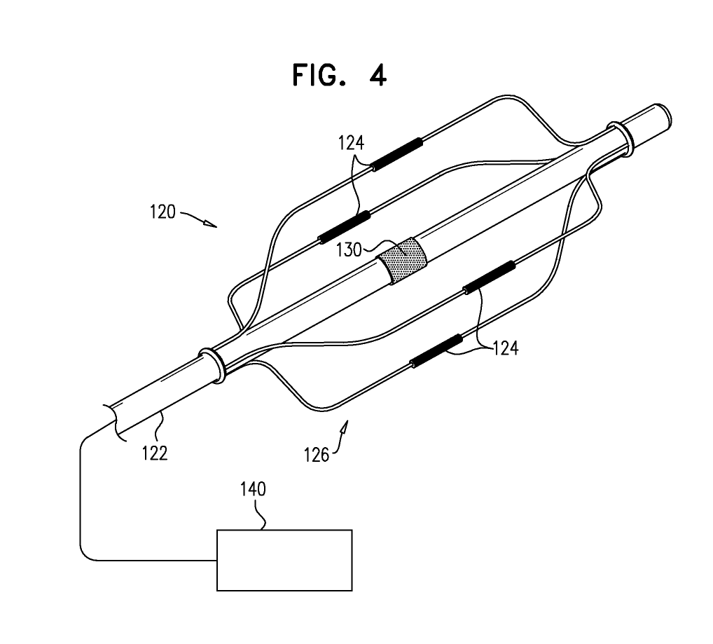

Reference is made to FIG. 4, which is a schematic illustration of an intravascular tool 120, in accordance with some applications of the invention. Tool 120 is used to perform techniques in which the techniques described with reference to FIGS. 1-3 are modified to utilize ultrasound-based denervation. Tool 120 comprises a longitudinal member (e.g., a catheter) 122 that has a distal portion that comprises at least one electrode 124, and is transluminally advanceable into a blood vessel of a subject, such as the renal artery of the subject. For some applications, tool 120 also comprises a blood pressure sensor (not shown), such as an intravascular pressure sensor, e.g., disposed on catheter 122, e.g., as described for system 80, mutatis mutandis. Alternatively, pressure sensor 98 may be an extracorporeal blood pressure sensor, such as a blood pressure cuff. Tool 120 is typically used in combination with a control unit 140 to which it is coupled. Control unit 140 is similar to control unit 100, mutatis mutandis.

Tool 120 further comprises at least one ultrasound transducer 130, which is positioned and oriented with respect to electrodes 124 such that it applies ultrasound to the same tissue site to which electrodes 124 apply the excitation component. For example, and as shown, transducer 130 may be positioned at the same longitudinal position of tool 120 as electrodes 124. Typically, tool 120 comprises a reversibly-expandable electrode device 126 on which the at least one electrode 124 is mounted. Whereas electrodes 124 require contact with the blood vessel wall in order to apply current (e.g., the excitatory component) to the nerve, transducer 130 is typically disposed on catheter 122, and is not placed in contact with the blood vessel wall. For some applications, and as shown, device 126 positions a plurality of electrodes 124 circumferentially around, and longitudinally aligned with, transducer 130. For such applications, transducer 130 typically applies ultrasound in one or more circumferential arcs that include the electrodes (e.g., in 360 degrees). Advantageously, when device 126 is expanded, it supports transducer 130 away from the wall of the blood vessel.

It is to be understood that for some applications, other shapes and positions of transducer 130 are used.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method, comprising:
during a dual-signal period, applying, via an electrode disposed at a site of a nerve of a subject, a dual electric signal that has (i) an excitation component that has a frequency of 20-500 Hz, and (ii) an ablation component that has a frequency of 5 kHz-1 GHz; and
during an ablation-signal period that is separate from the dual-signal period, applying, via the electrode disposed at the site, an ablation electric signal that has the ablation component but not the excitation component.

2. The method according to claim 1, wherein the dual electric signal is a dual electric signal that alternates between the excitation component and the ablation component more frequently than once every two seconds, and wherein applying the dual electric signal during the dual-signal period comprises applying, during the dual-signal period, the dual electric signal that alternates between the excitation component and the ablation component more frequently than once every two seconds.

3. The method according to claim 1, wherein the dual electric signal is a complex signal that contains the excitation component and the ablation component, and wherein applying the dual electric signal during the dual-signal period comprises applying, during the dual-signal period, the complex signal that contains the excitation component and the ablation component.

4-7. (canceled)

8. The method according to claim 1, further comprising detecting a change in a value of a parameter of a subject during the first dual-signal period, and at least in part responsively to the detected change, configuring one or more characteristics selected from the group consisting of: a duration of the ablation-signal period, a frequency of the ablation electric signal, and an amplitude of the ablation electric signal.

9. The method according to claim 1, wherein:
the dual-signal period is a first dual-signal period, and applying the dual electric signal comprises applying a first application of the dual electric signal,
the ablation-signal period is subsequent to the first dual-signal period, and
the method further comprises:
detecting a change in a value of a parameter of a subject during the first dual-signal period;
during a second dual-signal period that is subsequent to the ablation-signal period, applying, via the electrode disposed at the site, a second application of the dual electric signal; and
at least in part responsively to the detected change, configuring one or more characteristics selected from the group consisting of: a duration of the second dual-signal period, a frequency of the dual electric signal during the second application of the dual electric signal, and an amplitude of the dual electric signal during the second application of the dual electric signal.

10. The method according to claim 9, wherein the selected characteristic is the duration of the second dual-signal period, and configuring the one or more characteristics comprises configuring the duration of the second dual-signal period.

11. The method according to claim 10, wherein detecting the change in the value of the parameter comprises timing a duration of the first dual-signal period until the detected change reaches a pre-determined magnitude, and configuring the duration of second dual-signal period comprises configuring the duration of the second dual-signal period according to the duration of the first dual-signal period.

12. The method according to claim 1, wherein:
the ablation-signal period is subsequent to the dual-signal period,
the method further comprises detecting a change in a value of a parameter of a subject during the dual-signal period, and
applying the ablation electric signal comprises applying the ablation electric signal in response to the change in the value of the parameter during the dual-signal period.

13. The method according to claim 12, wherein:
the dual-signal period is a first dual-signal period, and applying the dual electric signal comprises applying a first application of the dual electric signal,
the ablation-signal period is a first ablation-signal period, and applying the ablation electric signal comprises applying a first application of the ablation electric signal, and
the method further comprises:
during a second dual-signal period that is subsequent to the first ablation-signal period, applying, via the electrode disposed at the site, a second application of the dual electric signal;
detecting a change in the value of the parameter during the second dual-signal period; and
in response to the change in the value of the parameter during the second dual-signal period, during a second ablation-signal period, applying, via the electrode disposed at the site, a second application of the ablation electric signal.

14. The method according to claim 13, wherein applying the second application of the ablation electric signal in response to the change in the value of the parameter during the second dual-signal period comprises applying the second application of the ablation electric signal in response to a difference between (i) the change in the value of the parameter during the first dual-signal period, and (ii) the change in the value of the parameter during the second dual-signal period.

15. The method according to claim 12, wherein:
the dual-signal period is a first dual-signal period, and applying the dual electric signal comprises applying a first application of the dual electric signal,
the ablation-signal period is a first ablation-signal period, and applying the ablation electric signal comprises applying a first application of the ablation electric signal, and
the method further comprises:
during a second dual-signal period that is subsequent to the ablation-signal period, applying, via the electrode disposed at the site, a second application of the dual electric signal;
detecting a change in the value of the parameter during the second dual-signal period; and
in response to the change in the value of the parameter during the second dual-signal period, moving the electrode to another site of the nerve of the subject.

16. The method according to claim 15, wherein moving the electrode to the other site of the nerve of the subject in response to the change in the value of the parameter during the second dual-signal period comprises moving the electrode to the other site of the nerve of the subject in response to a difference between (i) the change in the value of the parameter during the first dual-signal period, and (ii) the change in the value of the parameter during the second dual-signal period.

17. Apparatus for use with a blood vessel of a subject, the apparatus comprising:
a longitudinal member that has a proximal portion and a distal portion, comprising an electrode disposed at the distal portion, the distal portion being transluminally advanceable to the blood vessel; and
a control unit, in electrical communication with the electrode, and comprising circuitry, configured to:
during a dual-signal period, drive the electrode to apply a dual electric signal that has (i) an excitation component that has a frequency of 20-500 Hz, and (ii) an ablation component that has a frequency of 5 kHz-1 GHz; and
during an ablation-signal period that is separate from the dual-signal period, drive the electrode to apply an ablation electric signal that has the ablation component but not the excitation component.

18. The apparatus according to claim 17, wherein the dual electric signal is a dual electric signal that alternates between the excitation component and the ablation component more frequently than once every two seconds, and wherein the control unit is configured to, during the dual-signal period, drive the electrode to apply the dual electric signal that alternates between the excitation component and the ablation component more frequently than once every two seconds.

19. The apparatus according to claim 17, wherein the dual electric signal is a complex signal that contains the excitation component and the ablation component, and wherein the control unit is configured to, during the dual-signal period, drive the electrode to apply the complex signal that contains the excitation component and the ablation component.

20-23. (canceled)

24. The apparatus according to claim 17, wherein the apparatus is for use with a blood pressure sensor, and wherein:
the control unit comprises a blood-pressure-sensor interface that provides communication between the control unit and the blood pressure sensor; and
the circuitry is configured to receive blood-pressure information from the blood pressure sensor during the dual-signal period.

25. (canceled)

26. The apparatus according to claim 24, wherein:
the dual-signal period is a first dual-signal period, and the dual electric signal applied during the first dual-signal period is a first application of the dual electric signal, and
the circuitry is configured:
such that the ablation-signal period is subsequent to the first dual-signal period,
to apply, during a second dual-signal period that is subsequent to the ablation-signal period, a second application of the dual electric signal,
to identify a change in a blood-pressure value of the subject during the first dual-signal period,
at least in part responsively to the change, to configure one or more characteristics selected from the group consisting of: a duration of the second dual-signal period, a frequency of the dual electric signal during the second application of the dual electric signal, and an amplitude of the dual electric signal during the second application of the dual electric signal.

27. The apparatus according to claim 24, wherein the circuitry is configured to identify a change in a blood-pressure value of the subject during the first dual-signal period, and at least in part responsively to the change, to configure one or more characteristics selected from the group consisting of: a duration of the ablation-signal period, a frequency of the ablation electric signal, and an amplitude of the ablation electric signal.

28. A method, comprising:
advancing an electrode into a blood vessel of a subject; and
while the electrode is disposed at a site within the blood vessel, activating a control unit to drive the electrode to apply a dual electric signal that has (i) an excitation component that has a frequency of 20-500 Hz, and (ii) an ablation component that has a frequency of 5 kHz-1 GHz, and that alternates between the excitation component and the ablation component more frequently than once every two seconds.

29-34. (canceled)

35. The method according to claim 28, wherein:
the dual electric signal alternates between bursts of the excitation component and bursts of the ablation component such that, during a dual-signal period (i) in which the dual signal is applied, (ii) that has a duration and, (iii) that contains an equal number of excitation-component bursts and ablation-component bursts, the ablation-component bursts cumulatively have an ablation-component duration of 70-95 percent of a duration of the dual-signal period, and
activating the control unit to apply the dual electric signal comprises activating the control unit to apply the dual electric signal that alternates between bursts of the excitation component and bursts the ablation component such that, during the dual-signal period, the ablation-component bursts cumulatively have the ablation-component duration of 70-95 percent of the duration of the dual signal period.

36. The method according to claim 28, wherein:
the dual electric signal alternates between bursts of the excitation component and bursts of the ablation component such that, during a dual-signal period that has a duration and contains an equal number of excitation-component bursts and ablation-component bursts, the excitation-component bursts cumulatively have an excitation-component duration of 5-30 percent of a duration of the dual-signal period, and
activating the control unit to apply the dual electric signal comprises activating the control unit to apply the dual electric signal that alternates between bursts of the excitation component and bursts of the ablation component such that, during the dual-signal period, the excitation-component bursts cumulatively have an excitation-component duration of 5-30 percent of the duration of the dual signal period.

37-46. (canceled)
